(19) United States
(12) Patent Application Publication
(10) Pub. No.: US 2026/0015310 A1
(43) Pub. Date: Jan. 15, 2026

(54) Crude terephthalic acid hydrorefining catalyst, preparation method therefor, and application thereof

(71) Applicants: CHINA PETROLEUM & CHEMICAL CORPORATION, Beijing (CN); SINOPEC (SHANGHAI) RESEARCH INSTITUTE OF PETROCHEMICAL TECHNOLOGY CO., LTD., Shanghai (CN)

(72) Inventors: Zhongbin XIAO, Shanghai (CN); Xiaoli ZHU, Shanghai (CN); Wei LUO, Shanghai (CN); Guangbin SUN, Shanghai (CN); Limin HE, Shanghai (CN); Xiaochen GAO, Shanghai (CN)

(21) Appl. No.: 18/992,878

(22) PCT Filed: Jul. 7, 2023
(86) PCT No.: PCT/CN2023/106209

§ 371 Date: Jan. 9, 2025

(30) Foreign Application Priority Data
Jul. 12, 2022 (CN) 202210822061.0

(51) Int. Cl. C07C 63/26; B01J 23/44; B01J 23/46; B01J 35/00; B01J 35/30; B01J 35/53; B01J 37/08; B01J 37/16; C07C 51/50

(52) U.S. Cl. CPC C07C 63/26; B01J 23/44; B01J 23/462; B01J 35/19; B01J 35/397; B01J 35/53; B01J 37/08; B01J 37/16; C07C 51/50

(57) ABSTRACT

A catalyst for hydrorefining crude terephthalic acid, as well as its preparation method and application are provided. The catalyst includes a support and active components. The active components contains palladium and ruthenium in a weight ratio of (3-10):1, on element basis. The palladium is Pd⁰, and ruthenium includes Ru⁰ and Ru⁴⁺. Ru⁴⁺ and Ru⁰ are in a weight ratio of 0.1-1.0. The catalyst may be particularly suitable for hydrorefining crude terephthalic acid.

### TECHNICAL FIELD

The present disclosure relates to catalysts for hydrorefining crude terephthalic acid. In particular, the present disclosure relates to supported catalysts for hydrorefining crude terephthalic acid which comprise multiple active components, as well as their preparation methods and applications.

### BACKGROUND

P-terephthalic acid (or terephthalic acid, TA) is an important industrial raw material. It is used in producing polyethylene terephthalate (PET), which is then used to produce products such as fibers, bottles, films, and the like. Terephthalic acid can also be used in producing polybutylene terephthalate (PBT) engineering plastics, and the like. At present, terephthalic acid is mainly produced in a liquid-phase oxidation by using p-xylene as a raw material, acetic acid as a solvent, air as an oxidant, and Co/Mn/Br as a catalyst system. The resulting product after the reaction may contain by-products and impurities, and is generally referred to as Crude Terephthalic Acid (CTA). The by-products may include p-Toluyl alcohol (TALC), p-Tolualdehyde (TALD), p-Toluic acid (p-Toluylic acid), p-carboxybenzaldehyde (4-CBA), and the like. In the subsequent procedures for preparing polyesters, the presence of 4-CBA may have an adverse effect on the color and quality of the obtained polyesters. Crude terephthalic acid typically contains up to 9000 ppm of 4-CBA, which makes it unable to meet the standards for producing polyesters. Many methods have been proposed to remove impurities and by-products, especially 4-CBA, from crude terephthalic acid, to obtain purified terephthalic acid (PTA). Purified terephthalic acid is generally referred to purified products containing at least lower amount of 4-CBA than crude terephthalic acid. Purified terephthalic acid qualified for polymerizations may contain 25 ppm or less of 4-CBA. 4-CBA in crude terephthalic acid may usually be removed via hydrorefining, i.e. converting it into other compounds (such as p-Toluic acid) through hydrogenation, and then separating and purifying by crystallization. The hydrorefining of crude terephthalic acid may usually use palladium/carbon catalysts.

A single active component is used in palladium/carbon catalysts. Accordingly, the distribution of palladium metal on the support may have deep impact on performances of the catalysts. On the one hand, hydrorefining is a first-order reaction and has a fast reaction rate. In this regard, it is difficult for reactant molecules to penetrate into the interior of catalyst particles during the reaction. It means that the active component inside the particles may not be able to contact with the reactant molecules due to steric hindrance, and thereby cannot function. For such cases, only the active component on the outer surface exhibits catalytic activity. In order to fully utilize the active component (palladium), palladium/carbon catalysts are usually made to have a shell (i.e. in a core-shell structure), where the active component (palladium) is mainly loaded on the surface of the support. Due to the fact that the active component (palladium) of core-shell catalysts is mainly concentrated on the surface of the support, the catalysts may have a larger surface area in contact with reactant molecules and thereby more efficient catalytic ability than those catalysts wherein the same amount of active component is dispersed throughout the support. On the other hand, hydrorefining is usually operated under conditions of a reaction pressure of 6.5-8.5 MPa and a reaction temperature of 250-290° C. Grain growth of the active component (palladium) is inevitable under such reaction conditions. One of the main reasons for catalyst deactivation is just the grain growth of palladium. A deactivated commercial palladium/carbon catalyst for hydrorefining crude terephthalic acid may have palladium grains grown to 20 nm or bigger, whereas the corresponding fresh catalyst may have palladium grains of 2-5 nm. The faster the grain growth of palladium, the shorter the service life of the catalyst, resulting in significant economic losses.

Many methods have been proposed to improve the life of catalysts for hydrorefining crude terephthalic acid, including, for example, to use multiple active components. For example, U.S. Pat. No. 4,892,972 discloses a double-layer catalyst using Pd/C and Rh/C, with Pd and Rh in a ratio of 10:1. When used for hydrorefining crude terephthalic acid, the life of the catalyst was found significantly improved. Though Rh grains are not easy to grow, the price of Rh is ten times that of Pd. Therefore, such catalyst is not applicable in practice. Also suggested is to use a supported bimetallic catalyst containing Pd and Ru. In the preparation of such catalyst, Ru source cannot be easily reduced to metal Ru. In addition, such catalyst may require titanium dioxide and the like as the support. Those types of support may have poor acid and alkali resistance and may not be suitable for the reaction conditions for hydrorefining crude terephthalic acid.

Improvements on the activity and thermal stability of catalysts for hydrorefining crude terephthalic acid have been achieved in the art. However, there is always a demand for further improvements on the activity and thermal stability of catalysts for hydrorefining crude terephthalic acid.

### SUMMARY OF THE INVENTION

The present disclosure is to solve the problem that the hydrofining reaction of crude terephthalic acid in the prior art may suffer from low conversion of 4-CBA and poor thermal stability of catalysts. In this regard, provided in the present disclosure is a new catalyst for hydrofining crude terephthalic acid, which comprises multiple active components. The catalyst in accordance with the present disclosure may have outstanding catalytic activity and anti-sintering performance, which may be particularly suitable for hydrorefining crude terephthalic acid, wherein the catalyst in accordance with the present disclosure may make the hydrorefining more efficient and stable. The present disclosure also relates to a method for preparing the catalyst and use of the catalyst in hydrorefining crude terephthalic acid.

In one aspect, provided in the present disclosure is a catalyst for hydrorefining crude terephthalic acid, comprising a support and active components;
wherein the active components comprise palladium and ruthenium;
wherein palladium and ruthenium are in a weight ratio of (3-10):1, on element basis;
wherein palladium is Pd⁰, and ruthenium comprises Ru⁰ and Ru⁴⁺; and
wherein Ru⁴⁺ and Ru⁰ are in a weight ratio of 0.1-1.0.

In a further aspect, provided in the present disclosure is a method for preparing the above catalyst for hydrorefining crude terephthalic acid, comprises steps of:
(1) providing a catalyst support;
(2) mixing the catalyst support of step (1) with sources of active metals and an alkylamine, wherein the obtained mixture is subjected to aging and a first heat treatment to obtain a catalyst precursor;
(3) reducing the catalyst precursor of step (2) with a reducing agent to obtain the catalyst.

In a further more aspect, provided in the present disclosure is use of the above catalyst for hydrorefining crude terephthalic acid in a hydrorefining reaction of crude terephthalic acid.

In particular, the present disclosure may include the following items.

1. A catalyst for hydrorefining crude terephthalic acid, comprising a support and active components;
wherein the active components are metal palladium and metal ruthenium, and the metal palladium and the metal ruthenium are in a weight ratio of (3-10):1;
wherein valence state of the metal palladium is Pd⁰, and valence state of the metal ruthenium comprises Ru⁰ and Ru⁴⁺.

2. The catalyst of item 1, characterized in that, in the catalyst, Ru⁴⁺ and Ru⁰ are present in a weight ratio of 0.1-1.0.

3. The catalyst of item 1, characterized in that, the support is an activated carbon; preferably, the activated carbon is at least one selected from the group consisting of a coal based activated carbon, a wood activated carbon and a nut shell activated carbon.

4. A method for preparing the catalyst of anyone of items 1-3, comprises steps of:
(1) pretreating an activated carbon to obtain a catalyst support;
(2) mixing active metals, an alkylamine and a solvent to obtain a catalyst precursor i;
(3) mixing the catalyst support of step (1) with the catalyst precursor i of step (2), and subjecting to aging, desolvating and thermal treating, to obtain a catalyst precursor ii;
(4) reducing the catalyst precursor ii of step (3) with a reducing agent, and subjecting to thermal treating, to obtain the catalyst.

5. The method of item 4, characterized in that, alkyl group of the alkylamine in step (2) is selected from the group consisting of C3-C20 alkyl.

6. The method of item 4, characterized in that, the active metals in step (2) are palladium and ruthenium; wherein sources of palladium and sources of ruthenium are palladium salts and ruthenium salts; wherein the palladium salts are at least one selected from the group consisting of palladium nitrate, palladium acetate, chloropalladic acid and salts thereof, and tetraammine dichloropalladium, preferably palladium acetate; and wherein the ruthenium salts are at least one selected from the group consisting of ruthenium nitrate, ruthenium acetate, and ruthenium trichloride, preferably ruthenium acetate.

7. The method of item 4, characterized in that, in step (2), the solvent, the alkylamine and the active metals are in a mass ratio of (5000-30000):(20-50):(5-20); wherein, as the active metals, palladium and ruthenium are in a mass ratio of (3-6):1.

8. The method of item 4, characterized in that, in step (3), the catalyst support of step (1) and the catalyst precursor i of step (2) are in a mass ratio of 1:(2-5).

9. The method of item 4, characterized in that, the reducing agent in step (4) is at least one selected from the group consisting of hydrogen, hydrazine hydrate, formaldehyde, formic acid, salts of formaldehyde or formates, preferably hydrazine hydrate.

10. Use of the catalyst of anyone of items 1-3 in a hydrorefining reaction of crude terephthalic acid, by subjecting crude terephthalic acid to the hydrorefining reaction in the presence of the catalyst, to obtain a purified terephthalic acid.

As compared with the prior art, the present disclosure may achieve the following advantages.

(1) The catalyst for hydrorefining crude terephthalic acid in accordance with the present disclosure comprises Pd and Ru as active components. In particular, the weight ratio of Ru⁴⁺ and Ru⁰ is controlled in a specific range of (0.1-1.0). Accordingly, good anti-sintering performance is obtained.

(2) The method for preparing the catalyst for hydrorefining crude terephthalic acid in accordance with the present disclosure is simple to operate. By selecting raw materials and processing conditions, Ru⁴⁺ and Ru⁰ are introduced into the catalyst and their weight ratio is effectively controlled, ensuring the obtaining of the above catalyst for hydrorefining crude terephthalic acid.

(3) When the catalyst in accordance with the present disclosure is used in the hydrorefining reaction of crude terephthalic acid, besides ensuring the catalytic performance, it shows advantage of high thermal stability, thereby achieving outstanding technical effects.

### DESCRIPTION OF THE DRAWINGS

FIG. 1 is an XPS pattern showing Pd⁰ in 3d zone in the catalyst prepared in Example 1;

FIG. 2 is an XPS pattern showing Ru⁴¹ in 3p zone in the catalyst prepared in Example 1.

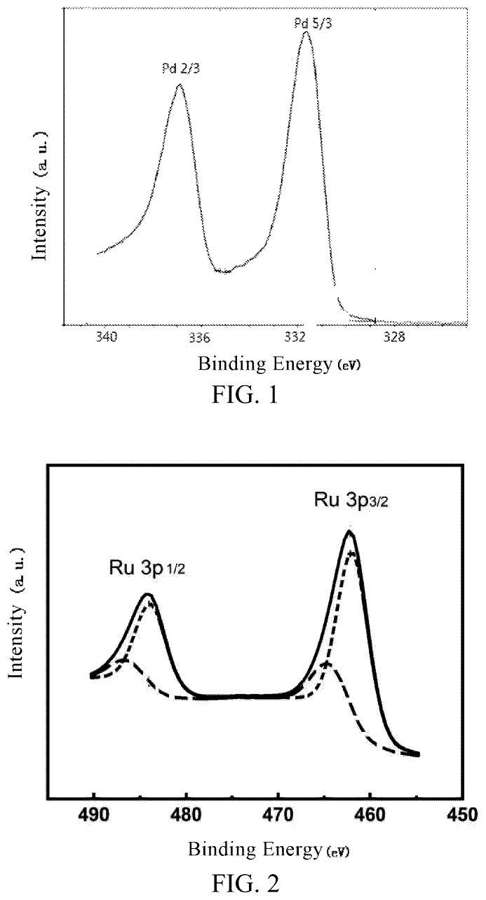

### DETAILED DESCRIPTION

Other than in the examples, all numerical values of parameters in this specification are to be understood as being modified in all instances by the term “about” whether or not “about” actually appears before the numerical value.

In one aspect, the present disclosure relates to a catalyst for hydrorefining crude terephthalic acid, comprising a support and active components;
wherein the active components comprise palladium and ruthenium;
wherein palladium and ruthenium are in a weight ratio of (3-10):1, on element basis;
wherein palladium is Pd⁰, and ruthenium comprises Ru⁰ and Ru⁴⁺; and
wherein Ru⁴⁺ and Ru⁰ are in a weight ratio of 0.1-1.0.

As used herein, the term “crude terephthalic acid” refers to a terephthalic acid product containing a relatively high concentration of p-carboxybenzaldehyde (4-CBA). In a variation, crude terephthalic acid contains, for example, at least 1000 ppm, at least 2500 ppm, at least 5000 ppm or at least 8000 ppm of p-carboxybenzaldehyde. As used herein, the term “hydrorefining crude terephthalic acid” refers to a process of converting and eliminating p-carboxybenzaldehyde by reacting crude terephthalic acid with hydrogen. Accordingly, a product having a reduced content of p-carboxybenzaldehyde in relative to crude terephthalic acid is referred to as “purified terephthalic acid”. For example, purified terephthalic acid may have a content of p-carboxybenzaldehyde of at least 100 ppm, at least 500 ppm, at least 1000 ppm, at least 2500 ppm, at least 5000 ppm or at least 8000 ppm lower than that of crude terephthalic acid. In a variation, purified terephthalic acid may have a content of p-carboxybenzaldehyde of 25 ppm or less, so that it can be directly used as a raw material for synthesizing polyesters, such as polyethylene terephthalate (PET).

In one embodiment, the catalyst for hydrorefining crude terephthalic acid may comprise 0.3-1 wt % of the active components. The active components may comprise palladium and ruthenium, wherein palladium and ruthenium are in a weight ratio of (3-10):1, preferably (3-6):1, on element basis. In a variation, palladium is Pd⁰, and ruthenium comprises Ru⁰ and Ru⁴⁺; and wherein Ru⁴⁺ and Ru⁰ are in a weight ratio of 0.1-1.0, preferably 0.2-0.8.

There is not special limitation in the present disclosure on the distribution of the active components on the support. In one embodiment, at least 50%, at least 75%, at least 90%, or at least 95% of the active components may be dispersed on the surface of the support. Preferably, the catalyst for hydrorefining crude terephthalic acid may have a core-shell structure, wherein the core comprises the support or is essentially consisting of the support, and the shell comprises the active components or is essentially consisting of the active components. In a variation, the catalyst for hydrorefining crude terephthalic acid may have a core-shell structure, wherein the core is essentially consisting of the support, and the shell is essentially consisting of palladium and ruthenium, and wherein palladium and ruthenium are uniformly distributed in the shell. Preferably, the shell may have a thickness of 10-200, preferably 40-100 microns.

In one embodiment, the support may be those commonly used in the art in a catalyst for hydrorefining crude terephthalic acid. In a variation, the support is an activated carbon. Preferably, the activated carbon is at least one selected from the group consisting of a coal based activated carbon, a wood activated carbon and a nut shell activated carbon. Preferably, the nut shell activated carbon is a coconut shell activated carbon. In a variation, the coconut shell activated carbon may have a specific surface area of 800-1600 m²/g and a pore volume of 0.35-0.80 mL/g. The coconut shell activated carbon is commercially available as particles (e.g., particles with a size of 4-8 mesh).

In a further aspect, the present disclosure relates to a method for preparing the above catalyst for hydrorefining crude terephthalic acid, comprises steps of:
(1) providing a catalyst support;
(2) mixing the catalyst support of step (1) with sources of active metals and an alkylamine, wherein the obtained mixture is subjected to aging and a first heat treatment to obtain a catalyst precursor;
(3) reducing the catalyst precursor of step (2) with a reducing agent to obtain the catalyst.

In one embodiment, step (1) of providing a catalyst support is operated by pretreating an activated carbon to obtain the catalyst support. The activated carbon may be pretreated by any conventional measure in the art to obtain the catalyst support. In one embodiment, the pretreatment includes washing and drying. In a variation, the washing is operated with water, wherein water and the activated carbon are in a volume ratio of (2-10):1; and the drying is operated at a temperature of 100-130° C. for 4-8 hours.

In one embodiment, before mixing the catalyst support of step (1) with the sources of active metals and the alkylamine, the sources of active metals and the alkylamine may be mixed with a solvent. In a variation, the solvent, the alkylamine and the sources of active metals are mixed in a mass ratio of (5000-30000):(20-50):(5-20). The obtained mixture may be mixed with the catalyst support of step (1) in a mass ratio of (2-5):1.

The solvent may be any organic solvent commonly used in the art. In one embodiment, the solvent is at least one selected from the group consisting of diethyl ether, dimethyl ether, ethanol, isopropanol, and acetone, preferably diethyl ether.

In one embodiment, step (2) may further comprise removing the solvent before the first heat treatment. In a variation, the solvent removing may comprise evaporating and condensing to recover the solvent, wherein the evaporation temperature is preferably 60-90° C.

In one embodiment, the sources of active metals may comprise sources of palladium and sources of ruthenium. In a variation, the sources of palladium are palladium salts and the sources of ruthenium are ruthenium salts. The suitable palladium salts may be at least one selected from the group consisting of palladium nitrate, palladium acetate, chloropalladic acid, chloropalladates, and tetraammine dichloropalladium, preferably palladium acetate. The suitable ruthenium salts may be at least one selected from the group consisting of ruthenium nitrate, ruthenium acetate, and ruthenium trichloride, preferably ruthenium acetate.

In one embodiment, the alkyl group in the alkylamine may be linear alkyl, preferably selected from the group consisting of C3-C20 linear alkyl groups, more preferably selected from the group consisting of C12-C18 linear alkyl groups. Examples of suitable alkylamines may include, but not limited to, dodecylamine, tetradecylamine, hexadecylamine and octadecylamine.

In one embodiment, the aging is operated for 2-24 hours, preferably 4-12 hours. There is not any special limitation in the present disclosure on the aging conditions. Those conditions commonly used in the art may be used. In a variation, the aging is operated under an oxygen-containing atmosphere, such as in air.

The first heat treatment may comprise heat treating at 150-250° C. for 2-8 hours under an inert atmosphere. The inert atmosphere may comprise nitrogen or an inert gas, preferably nitrogen.

In one embodiment, the reducing agent is at least one selected from the group consisting of hydrazine hydrate, formaldehyde, formic acid, salts of formaldehyde or formates, preferably hydrazine hydrate. In a variation, hydrazine hydrate and the catalyst precursor of step (2) are used in a mass ratio of 1:(2-10) to reduce the catalyst precursor of step (2) with hydrazine hydrate. Preferably, the reducing is operated at room temperature (25° C.) for 4-12 hours, preferably 6-9 hours.

In one embodiment, after reducing the catalyst precursor of step (2) with the reducing agent, the obtained catalyst may be subjected to a second heat treatment. The second heat treatment may comprise heat treating at 100-200° C. for 2-8 hours under an inert atmosphere. The inert atmosphere may comprise nitrogen or an inert gas, preferably nitrogen.

In a further more aspect, the present disclosure relates to use of the above catalyst for hydrorefining crude terephthalic acid in a hydrorefining reaction of crude terephthalic acid. Crude terephthalic acid may be subjected to the hydrorefining reaction in the presence of the catalyst, to obtain a purified terephthalic acid. In a variation, the hydrorefining reaction of crude terephthalic acid may be operated under reaction conditions including a reaction temperature of 250-350° C., preferably 270-290° C., and a reaction pressure of 6.5-8.5 MPa. During the hydrorefining reaction of crude terephthalic acid, 4-CBA may be converted and removed, obtaining the purified terephthalic acid.

The catalyst for hydrorefining crude terephthalic acid in accordance with the present disclosure may have a conversion for 4-CBA of at least 85%, preferably at least 90%, more preferably 95%, and most preferably 99%. The catalyst for hydrorefining crude terephthalic acid in accordance with the present disclosure may effectively inhibit the grain growth of palladium in the catalyst at high temperatures (e.g., 300° C. or higher, 400° C. or higher, and 500° C. or higher). In one embodiment, the catalyst for hydrorefining crude terephthalic acid in accordance with the present disclosure may have a grain growth rate for palladium in the catalyst of 25% or less, preferably 10% or less, and more preferably 5% or less at, for example, 300° C., 400° C., or 500° C. For example, the grain growth rate is 25%, 20%, 15%, 10%, 5%, 2% or 1%. In a variation, at 300° C., the grain growth rate for palladium in the catalyst for hydrorefining crude terephthalic acid in accordance with the present disclosure may be 10% or less, preferably 5% or less, and more preferably 3% or less. In general, the hydrorefining reaction is operated under conditions including a reaction pressure of 6.5-8.5 MPa and a reaction temperature of 250-290° C. Therefore, under conventional conditions for hydrorefining reaction, the catalyst for hydrorefining crude terephthalic acid in accordance with the present disclosure may effectively control the grain growth of palladium and thereby have excellent anti-sintering performance. Therefore, the catalyst for hydrorefining crude terephthalic acid in accordance with the present disclosure may make the hydrorefining reaction of crude terephthalic acid more efficient and stable.

### EXAMPLES

The features and advantages of the invention will become apparent from the following examples. The examples are intended to illustrate and not to limit the invention in any way.

### Methods for Testing

The contents of Pd and Ru in catalysts of the examples and the comparative examples were determined via ICP-AES.

The contents of Ru in different valence states in catalysts of the examples and the comparative examples were analyzed via XPS by using an ESCA-IAB MK II photoelectron spectrometer. The testing was operated under conditions of: a laser source of MgKa rays (hv-1486.6 eV), an operating voltage of 10 kV, an X-ray current of 20 mA, and contaminated carbon C_{1S} (Eb=284.6 eV) as energy correction. Under those conditions, the pattern of a catalysts was obtained, wherein there were a characteristic peak at 461.5 ev (Ru3p3/₂) corresponding to Ru⁰, and a characteristic peak at 465.2 ev (Ru3p3/₂) corresponding to Ru⁺⁴. The Ru3p3/₂ peaks were fitted and separated using the software XPS peakfit 4.1, and then the percentage contents of ruthenium in different valence states were calculated using the following formula.
the percentage content by atom number of ruthenium in certain valence

$$state = \text{?}$$

$$\text{?} \text{indicates text missing or illegible when filed}$$

wherein x was Ru in the valence state being analyzed; I was the area of the photoelectron peak; n was the number of the valence states of Ru being considered; S was a sensitivity factor.

The thermal stability of catalysts of the examples and the comparative examples were tested as follow.

The catalyst was calcined at 300° C., 400° C. and 500° C., respectively, for 8 hours under nitrogen atmosphere, and then cooled to room temperature. The calcined catalyst was detected by X-ray diffractometer (XRD), wherein the average grain size of palladium contained therein was calculated by using the Debye-Scherrer formula.

Debye-Scherrer formula: Dhkl=kλ/β cos θ
wherein Dhkl was the grain diameter along the direction perpendicular to the crystal plane (hkl), k was the Scherrer constant (usually 0.89), λ was the wavelength of the incident X-ray (wherein the wavelength of Cuka was 0.15406 nm, and the wavelength of Cuka1 was 0.15418 nm), θ was the Bragg diffraction angle (°), and β was the half-peak width of the diffraction peak (rad).

The thermal stability of the catalyst was expressed by the grain growth rate of the active components in the catalyst before and after the calcining. The larger the value, the lower the stability, and vice versa. The grain growth rate was calculated according to the following formula.

The grain growth rate=[(the average grain size of Pd after the calcining−the average grain size of Pd in the fresh catalyst)/the average grain size of Pd in the fresh catalyst]×100%

The activity of catalysts of the examples and the comparative examples were tested as follow.

Crude terephthalic acid was subjected to hydrorefining reaction under the conditions shown in the below table in the presence of catalysts of the examples and the comparative examples under nitrogen atmosphere, to obtain purified terephthalic acid. After being completely dissolved in aqueous ammonia, crude terephthalic acid and purified terephthalic acid were analyzed by high performance liquid chromatography (HPLC) to obtain their 4-CBA contents. Then, the conversion for 4-CBA was calculated to characterize the activity of the catalysts.

### Conditions for Hydrorefining Reaction:

| Reaction vessel | a 2 L stainless steel autoclave | |
|---|---|---|
| Amount of the catalyst | 2.0 | g |
| Amount of crude | 30.0 g (with a 4-CBA content of | |
| terephthalic acid | 10000 ppmw) | |
| Solvent | 1000 mL pure water | |
| Reaction pressure | 7.0 | MPa |
| Hydrogen partial pressure | 0.5 | MPa |
| Reaction time | 1.0 | hour |
| Reaction temperature | 280° | C. |

The conversion for 4-CBA=[(the 4-CBA content in the purified terephthalic acid−the 4-CBA content in crude terephthalic acid)/the 4-CBA content in crude terephthalic acid]×100%

### Example 1

100 g of flaky coconut shell activated carbon in a size of 4-8 mesh (with a specific surface area of 1100 m²/g, and a pore volume of 0.52 mL/g) was obtained and washed with pure water, wherein pure water and the activated carbon were in a volume ratio of 5:1, and then dried at 130° C. for 8 hours, to obtain a catalyst support. 400 g of a mixture of palladium acetate, ruthenium acetate, hexadecylamine and diethyl ether was stirred for 30 minutes, wherein Pd, Ru and hexadecylamine were in an amount of 1250 ppmw, 250 ppmw and 1.0 wt %, respectively. 100 g of the catalyst support was added to the mixture, wherein the catalyst support and the mixture were in a mass ratio of 1:4, and then the obtained mixture was subjected to aging for 8 hours, evaporating and condensing at 80° C. to recover diethyl ether, the first heat treating at 180° C. for 4 hours under a nitrogen atmosphere, and cooling to room temperature, to obtain a catalyst precursor. 200 g of hydrazine hydrate (with a concentration of 20 wt %) was added to the catalyst precursor, to operate the reducing for 8 hours. The mixture was then subjected to the second heat treatment at 180° C. for 4 hours under a nitrogen atmosphere and cooling to room temperature, to obtain a catalyst.

The contents of active components, the thermal stability and the activity of the catalyst were evaluated by the methods for testing described above. The results were listed in Table 1.

### Example 2

100 g of flaky coconut shell activated carbon in a size of 4-8 mesh (with a specific surface area of 1100 m²/g, and a pore volume of 0.52 mL/g) was obtained and washed with pure water, wherein pure water and the activated carbon were in a volume ratio of 5:1, and then dried at 130° C. for 8 hours, to obtain a catalyst support. 400 g of a mixture of palladium acetate, ruthenium acetate, hexadecylamine and diethyl ether was stirred for 30 minutes, wherein Pd, Ru and tetradecylamine were in an amount of 1250 ppmw, 250 ppmw and 1.0 wt %, respectively. 100 g of the catalyst support was added to the mixture, wherein the catalyst support and the mixture were in a mass ratio of 1:4, and then the obtained mixture was subjected to aging for 8 hours, evaporating and condensing at 80° C. to recover diethyl ether, the first heat treating at 180° C. for 4 hours under a nitrogen atmosphere, and cooling to room temperature, to obtain a catalyst precursor. 200 g of hydrazine hydrate (with a concentration of 20 wt %) was added to the catalyst precursor, to operate the reducing for 9 hours. The mixture was then subjected to the second heat treatment at 180° C. for 4 hours under a nitrogen atmosphere and cooling to room temperature, to obtain a catalyst.

The contents of active components, the thermal stability and the activity of the catalyst were evaluated by the methods for testing described above. The results were listed in Table 1.

### Example 3

100 g of flaky coconut shell activated carbon in a size of 4-8 mesh (with a specific surface area of 1100 m²/g, and a pore volume of 0.52 mL/g) was obtained and washed with pure water, wherein pure water and the activated carbon were in a volume ratio of 5:1, and then dried at 130° C. for 8 hours, to obtain a catalyst support. 400 g of a mixture of palladium acetate, ruthenium acetate, hexadecylamine and diethyl ether was stirred for 30 minutes, wherein Pd, Ru and hexadecylamine were in an amount of 1250 ppmw, 250 ppmw and 1.0 wt %, respectively. 100 g of the catalyst support was added to the mixture, wherein the catalyst support and the mixture were in a mass ratio of 1:4, and then the obtained mixture was subjected to aging for 8 hours, evaporating and condensing at 80° C. to recover diethyl ether, the first heat treating at 180° C. for 4 hours under a nitrogen atmosphere, and cooling to room temperature, to obtain a catalyst precursor. 200 g of hydrazine hydrate (with a concentration of 20 wt %) was added to the catalyst precursor, to operate the reducing for 7 hours. The mixture was then subjected to the second heat treatment at 180° C. for 4 hours under a nitrogen atmosphere and cooling to room temperature, to obtain a catalyst. The obtained catalyst was calcined at 300° C., 400° C. and 500° C., respectively, for 8 hours under nitrogen atmosphere to evaluate the thermal stability of the catalyst.

The contents of active components, the thermal stability and the activity of the catalyst were evaluated by the methods for testing described above. The results were listed in Table 1.

### Example 4

100 g of flaky coconut shell activated carbon in a size of 4-8 mesh (with a specific surface area of 1100 m²/g, and a pore volume of 0.52 mL/g) was obtained and washed with pure water, wherein pure water and the activated carbon were in a volume ratio of 5:1, and then dried at 130° C. for 8 hours, to obtain a catalyst support. 400 g of a mixture of palladium acetate, ruthenium acetate, hexadecylamine and diethyl ether was stirred for 30 minutes, wherein Pd, Ru and hexadecylamine were in an amount of 1250 ppmw, 250 ppmw and 1.0 wt %, respectively. 100 g of the catalyst support was added to the mixture, wherein the catalyst support and the mixture were in a mass ratio of 1:4, and then the obtained mixture was subjected to aging for 8 hours, evaporating and condensing at 80° C. to recover diethyl ether, the first heat treating at 180° C. for 4 hours under a nitrogen atmosphere, and cooling to room temperature, to obtain a catalyst precursor. 200 g of hydrazine hydrate (with a concentration of 20 wt %) was added to the catalyst precursor, to operate the reducing for 6 hours. The mixture was then subjected to the second heat treatment at 180° C. for 4 hours under a nitrogen atmosphere and cooling to room temperature, to obtain a catalyst.

The contents of active components, the thermal stability and the activity of the catalyst were evaluated by the methods for testing described above. The results were listed in Table 1.

### Comparative Example 1

100 g of flaky coconut shell activated carbon in a size of 4-8 mesh (with a specific surface area of 1100 m²/g, and a pore volume of 0.52 mL/g) was obtained and washed with pure water, wherein pure water and the activated carbon were in a volume ratio of 5:1, and then dried at 130° C. for 8 hours, to obtain a catalyst support. 400 g of a mixture of palladium acetate, hexadecylamine and diethyl ether was stirred for 30 minutes, wherein Pd and hexadecylamine were in an amount of 1250 ppmw and 1.0 wt %, respectively. 100 g of the catalyst support was added to the mixture, wherein the catalyst support and the mixture were in a mass ratio of 1:4, and then the obtained mixture was subjected to aging for 8 hours, evaporating and condensing at 80° C. to recover diethyl ether, the first heat treating at 180° C. for 4 hours under a nitrogen atmosphere, and cooling to room temperature, to obtain a catalyst precursor. 200 g of hydrazine hydrate (with a concentration of 20 wt %) was added to the catalyst precursor, to operate the reducing for 8 hours. The mixture was then subjected to the second heat treatment at 180° C. for 4 hours under a nitrogen atmosphere and cooling to room temperature, to obtain a catalyst. The contents of active components, the thermal stability and the activity of the catalyst were evaluated by the methods for testing described above. The results were listed in Table 1.

### Comparative Example 2

100 g of flaky coconut shell activated carbon in a size of 4-8 mesh (with a specific surface area of 1100 m²/g, and a pore volume of 0.52 mL/g) was obtained and washed with pure water, wherein pure water and the activated carbon were in a volume ratio of 5:1, and then dried at 130° C. for 8 hours, to obtain a catalyst support. 400 g of a mixture of palladium acetate, ruthenium acetate, hexadecylamine and diethyl ether was stirred for 30 minutes, wherein Pd, Ru and hexadecylamine were in an amount of 1250 ppmw, 250 ppmw and 1.0 wt %, respectively. 100 g of the catalyst support was added to the mixture, wherein the catalyst support and the mixture were in a mass ratio of 1:4, and then the obtained mixture was subjected to aging for 8 hours, evaporating and condensing at 80° C. to recover diethyl ether, and cooling to room temperature, to obtain a catalyst precursor. The catalyst precursor was subjected to the second heat treatment at 400° C. for 8 hours under a hydrogen atmosphere and cooling to room temperature, to obtain a catalyst.

The contents of active components, the thermal stability and the activity of the catalyst were evaluated by the methods for testing described above. The results were listed in Table 1.

### Comparative Example 3

100 g of flaky coconut shell activated carbon in a size of 4-8 mesh (with a specific surface area of 1100 m²/g, and a pore volume of 0.52 mL/g) was obtained and washed with pure water, wherein pure water and the activated carbon were in a volume ratio of 5:1, and then dried at 130° C. for 8 hours, to obtain a catalyst support. 400 g of a mixture of palladium acetate, ruthenium acetate, hexadecylamine and diethyl ether was stirred for 30 minutes, wherein Pd, Ru and hexadecylamine were in an amount of 1250 ppmw, 250 ppmw and 1.0 wt %, respectively. 100 g of the catalyst support was added to the mixture, wherein the catalyst support and the mixture were in a mass ratio of 1:4, and then the obtained mixture was subjected to aging for 8 hours, evaporating and condensing at 80° C. to recover diethyl ether, the first heat treating at 180° C. for 4 hours under a nitrogen atmosphere, and cooling to room temperature, to obtain a catalyst precursor. 200 g of hydrazine hydrate (with a concentration of 2 wt %) was added to the catalyst precursor, to operate the reducing for 3 hours. The mixture was then subjected to the second heat treatment at 180° C. for 4 hours under a nitrogen atmosphere and cooling to room temperature, to obtain a catalyst.

The contents of active components, the thermal stability and the activity of the catalyst were evaluated by the methods for testing described above. The results were listed in Table 1.

### Comparative Example 4

100 g of flaky coconut shell activated carbon in a size of 4-8 mesh (with a specific surface area of 1100 m²/g, and a pore volume of 0.52 mL/g) was obtained and washed with pure water, wherein pure water and the activated carbon were in a volume ratio of 5:1, and then dried at 130° C. for 8 hours, to obtain a catalyst support. 400 g of a mixture of palladium acetate, ruthenium acetate, hexadecylamine and diethyl ether was stirred for 30 minutes, wherein Pd, Ru and hexadecylamine were in an amount of 1250 ppmw, 250 ppmw and 1.0 wt %, respectively. 100 g of the catalyst support was added to the mixture, wherein the catalyst support and the mixture were in a mass ratio of 1:4, and then was subjected to aging for 8 hours, evaporating and condensing at 80° C. to recover diethyl ether, first heat treating at 180° C. for 4 hours under a nitrogen atmosphere, and cooling to room temperature, to obtain a catalyst precursor. 200 g of hydrazine hydrate (with a concentration of 20 wt %) was added to the catalyst precursor, to operate the reducing for 12 hours. The mixture was then subjected to second heat treatment at 180° C. for 4 hours under a nitrogen atmosphere and cooling to room temperature, to obtain a catalyst.

The content of active components, the thermal stability and the activity of the catalyst were evaluated by the methods for testing described above. The results were listed in Table 1.

### Comparative Example 5

100 g of flaky coconut shell activated carbon in a size of 4-8 mesh (with a specific surface area of 1100 m²/g, and a pore volume of 0.52 mL/g) was obtained and washed with pure water, wherein pure water and the activated carbon were in a volume ratio of 5:1, and then dried at 130° C. for 8 hours, to obtain a catalyst support. 400 g of a mixture of palladium acetate, ruthenium acetate and diethyl ether was stirred for 30 minutes, wherein Pd and Ru were in an amount of 1250 ppmw and 250 ppmw, respectively. 100 g of the catalyst support was added to the mixture, wherein the catalyst support and the mixture were in a mass ratio of 1:4, and then the obtained mixture was subjected to aging for 8 hours, evaporating and condensing at 80° C. to recover diethyl ether, and cooling to room temperature, to obtain a catalyst precursor. The catalyst precursor was subjected to the second heat treatment at 80° C. for 8 hours under a hydrogen atmosphere and cooling to room temperature, to obtain a catalyst.

The contents of active components, the thermal stability and the activity of the catalyst were evaluated by the methods for testing described above. The results were listed in Table 1.

### Comparative Example 6

The example 1 of CN102039123A was repeated herein.

50 g of granular coconut shell activated carbon support was pretreated by being soaked in 400 ml of 0.01% dilute nitric acid solution for 4 hours, then washed with deionized water until neutral, drained, dried at 105° C. for 24 hours, and naturally cooled to room temperature.

Chloropalladic acid, hydrated ruthenium trichloride and tartaric acid were dissolved in 20 ml of deionized water. A solution of hydroxymethyl cellulose in pure water was added thereto. The obtained solution was adjusted to a pH of 3.0 with 8% sodium carbonate, and finally adjusted to a volume of 40 ml, to obtain a palladium colloid. The palladium colloid comprised (by weight):

| | |
|---|---|
| Pd (from chloropalladic acid) | 0.48% |
| Ru (from hydrated ruthenium trichloride) | 0.02% |
| tartaric acid | 0.02% |
| hydroxymethyl cellulose | 0.01% |

The support was placed in a rotating plate, and the prepared palladium colloid was sprayed onto the support within 5 minutes. The support was placed for more than 8 hours, and then reduced with hydrogen at 200° C. for 6 hours, naturally cooled to room temperature under a hydrogen atmosphere, and finally washed with pure water until no Cl⁻ was present, to obtain a catalyst for hydrorefining terephthalic acid, Cat 1.

The contents of active components of Cat 1 were evaluated by the methods for testing described above. The results showed that the catalyst was consisting of palladium in a valence state of zero and ruthenium in a valence state of 3. Further, the thermal stability and the activity of Cat 1 were evaluated by the methods for testing described above. The results were listed in Table 1.

TABLE 1
test results of catalysts of examples and comparative examples

| | | | | | | After calcining | | After calcining | | After calcining | | |
|---|---|---|---|---|---|---|---|---|---|---|---|---|
| | | | | | | at 300° C. | | at 400° C. | | at 500° C. | | |
| | | | | | | (nm) | | (nm) | | (nm) | | |
| | | | | | Average | Average | | Average | | Average | | |
| | | | | | gain size | gain size | Grain | gain size | Grain | gain size | Grain | |
| | Pd | Ru | Weight | Weight | in fresh | in the | growth | in the | growth | in the | growth | Conversion |
| | content | content | ratio of | ratio of | catalyst | catalyst | rate | catalyst | rate | catalyst | rate | for 4-CBA |
| | (%) | (%) | Ru⁴⁺/Ru⁰ | Ru³⁺/Ru⁰ | (nm) | (nm) | (%) | (nm) | (%) | (nm) | (%) | (%) |
| Ex. 1 | 0.502 | 0.101 | 0.40/1 | 0 | 3.80 | 3.82 | 0.52 | 4.01 | 5.52 | 4.28 | 12.63 | 99.6 |
| Ex. 2 | 0.485 | 0.082 | 0.36/1 | 0 | 3.85 | 3.95 | 2.59 | 4.27 | 10.90 | 4.73 | 22.85 | 98.1 |
| Ex. 3 | 0.496 | 0.100 | 0.55/1 | 0 | 3.85 | 4.00 | 3.90 | 4.26 | 10.65 | 4.66 | 21.04 | 99.0 |
| Ex. 4 | 0.500 | 0.098 | 0.75/1 | 0 | 3.87 | 3.91 | 1.03 | 4.12 | 6.46 | 4.30 | 11.11 | 99.3 |
| CE. 1 | 0.501 | 0 | — | — | 3.95 | 4.27 | 8.10 | 6.53 | 65.31 | 12.6 | 218.99 | 65.8 |
| CE. 2 | 0.499 | 0.093 | +∞ | — | 4.01 | 4.25 | 5.98 | 5.82 | 45.13 | 10.8 | 169.32 | 70.4 |
| CE. 3 | 0.498 | 0.088 | 3.02/1 | 0 | 3.86 | 4.13 | 7.00 | 5.16 | 33.68 | 6.87 | 77.98 | 96.7 |
| CE. 4 | 0.497 | 0.091 | 0.08/1 | 0 | 3.90 | 4.16 | 6.67 | 5.37 | 37.69 | 7.09 | 81.79 | 95.4 |
| CE. 5 | 0.499 | 0.099 | — | +∞ | 3.99 | 4.24 | 6.27 | 6.47 | 62.16 | 12.1 | 203.26 | 63.5 |
| CE. 6 | 0.479 | 0.019 | — | +∞ | 3.50 | 4.18 | 19.42 | 6.48 | 85.14 | 12.4 | 254.29 | 63.3 |

1. A catalyst for hydrorefining crude terephthalic acid, comprising a support and active components;
wherein the active components comprise palladium and ruthenium;
wherein palladium and ruthenium are in a weight ratio of (3-10):1, on element basis;
wherein palladium is Pd⁰, and ruthenium comprises Ru⁰ and Ru⁴⁺; and
wherein Ru⁴⁺ and Ru⁰ are in a weight ratio of 0.1-1.0.

2. The catalyst as claimed in claim 1, characterized in that, the support is an activated carbon; preferably, the activated carbon is at least one selected from the group consisting of a coal based activated carbon, a wood activated carbon and a nut shell activated carbon.

3. The catalyst as claimed in claim 1, characterized in that, the catalyst has a core-shell structure, wherein the core is essentially consisting of the support, and the shell is essentially consisting of the active components;
preferably, the shell has a thickness of 10-200 microns;
preferably, palladium and ruthenium are uniformly distributed in the shell.

4. The catalyst as claimed in claim 1, characterized in that, the catalyst has a grain growth rate for palladium of 10% or less, preferably 5% or less, and more preferably 3% or less at 300° C.

5. A method for preparing the catalyst as claimed in claim 1, comprises steps of:
(1) providing a catalyst support;
(2) mixing the catalyst support of step (1) with sources of active metals and an alkylamine, wherein the obtained mixture is subjected to aging and a first heat treatment to obtain a catalyst precursor;
(3) reducing the catalyst precursor of step (2) with a reducing agent to obtain the catalyst.

6. The method as claimed in claim 5, characterized in that, the method further comprises: before mixing the catalyst support of step (1) with the sources of active metals and the alkylamine, mixing the sources of active metals and the alkylamine with a solvent, and
step (2) further comprises: removing the solvent before the first heat treatment.

7. The method as claimed in claim 5, characterized in that, the method further comprises: after step (3) of reducing, subjecting to a second heat treatment.

8. The method as claimed in claim 5, characterized in that, the alkyl group in the alkylamine is selected from the group consisting of C3-C20 alkyl groups.

9. The method as claimed in claim 5, characterized in that, the sources of active metals comprise sources of palladium and sources of ruthenium, preferably palladium salts and ruthenium salts, wherein the palladium salts are at least one selected from the group consisting of palladium nitrate, palladium acetate, chloropalladic acid, chloropalladate, and tetraammine dichloropalladium, preferably palladium acetate; and wherein the ruthenium salts are at least one selected from the group consisting of ruthenium nitrate, ruthenium acetate, and ruthenium trichloride, preferably ruthenium acetate.

10. The method as claimed in claim 6, characterized in that, in step (2), the solvent, the alkylamine and the active metals are in a mass ratio of (5000-30000):(20-50):(5-20).

11. The method as claimed in claim 5, characterized in that, the reducing agent is at least one selected from the group consisting of hydrazine hydrate, formaldehyde, formic acid, salts of formaldehyde and formates, preferably hydrazine hydrate;
preferably, hydrazine hydrate and the catalyst precursor of step (2) are used in a mass ratio of 1:(2-10) to reduce the catalyst precursor of step (2) with hydrazine hydrate;
preferably, the reducing is operated at room temperature for 4-12 hours, preferably 6-9 hours.

12. A method for hydrorefining of crude terephthalic acid, comprising contact the catalyst of claim 1 with crude terephthalic acid.
